(19) United States
(12) Patent Application Publication
(10) Pub. No.: US 2005/0053597 A1
(43) Pub. Date: Mar. 10, 2005

(54) Composition and method for treatment of wounds

(76) Inventor: David Idris Pritchard, Borrow Upon Soar Leics (GB)

Correspondence Address: JOHN S. PRATT, ESQ;KILPATRICK STOCKTON, LLP, 1100 PEACHTREE STREET, ATLANTA, GA, 30309, US

(21) Appl. No.: 10/495,740

(22) PCT Filed: Nov. 18, 2002
(86) PCT No.: PCT/GB02/05171

(30) Foreign Application Priority Data
Nov. 17, 2001 (GB) 0127618.7

(51) Int. Cl. A61K 38/48; A61K 38/16

(52) U.S. Cl. 424/94.63; 514/2

(57) ABSTRACT

A composition for treatment of a wound to promote healing thereof in a human or non-human mammal comprises an active amount of a toll receptor (or toll-like receptor) ligand, or a precursor thereof, and a suitable carrier. The ligand may be a spatzle or spatzle-like protein derived from an insect, such as Drosophila melanogaster or Lucilia sericata. The invention also relates to a method of treating a wound which comprises applying to the wound a composition according to the invention, and a dressing for a wound which comprises a support carrying composition according to the invention.

The present invention relates to the treatment of wounds. More particularly, it relates to substances which promote the healing of wounds, to compositions and to dressings which incorporate such substances and to a method of treating wounds using such substances.

Efficient wound healing is a complex physiological process which involves many mechanisms including cell migration, growth factor secretion, angiogenesis, tissue remodelling and the intrinsic proteinase/antiproteinase balance of the wound contributing in concert and in an apparently staged manner to accelerate controlled tissue regeneration.

Wound care products are essential in modern medical practice, especially for the treatment of patients with chronic wounds or burns. Many different substances have previously been proposed as having activities which contribute to the healing of wounds. These previously proposed substances include streptokinase, collagenase and streptodornase (all obtained from bacterial sources), bromelain (from pineapples), plasmin and trypsin (obtained from cattle) and krill enzymes (obtained from crustacea). Clinical trial data indicate that such substances are only partially effective in promoting the healing of wounds.

The larvae (maggots) of the green bottle fly, Lucilia sericata, are known to have significant wound healing attributes as live organisms. Debridement treatment using the larvae of Lucilia sericata, has become a widely accepted clinical practice. However, little has been reported in the literature about the way in which these larvae go about their task of cleaning wounds to an extent that conventionally untreatable wounds heal.

Although efficacious, live larvae are unpleasant to many patients and the use of live larvae on wounds and the introduction of their crude secretions into wounds, which inevitably occurs when the larvae are used, are unacceptable to many patients and to many medical practitioners. The use of live organisms also increases the risk of infection or allergic reactions in the patient.

### STATEMENTS OF INVENTION

Broadly, the invention relates to a composition for treatment of a wound to promote healing thereof in a human or non-human mammal which comprises an active amount of a toll receptor ligand, or a precursor thereof, and a suitable carrier. In this specification the term “toll receptor” should be taken as including the human and non-human homologues of the Drosophila toll receptor which are often referred to in the art as toll-like receptors (TLR's) and which represent a conserved family of innate immune recognition receptors which are coupled to a signalling pathway that is conserved in mammals, insects, and plants resulting in the activation of genes that mediate innate immune defences. Thus, for the avoidance of doubt, the term “toll receptor”, as used herein, means toll receptor and toll-like receptor and the term “toll receptor ligand”, as used herein, is to be construed accordingly. In this specification the term “ligand” should be taken to include the naturally produced ligands themselves, and any synthetic analogues thereof which would have the same function as the natural ligand. According to a particular embodiment of the present invention the ligand may be selected from constitutive or induced ligands for human toll receptors and which may or may not be processed by proteases, such as serine proteases.

Typically, the toll receptor ligand or ligand precursor is a member of the cysteine knot superfamily of proteins, or an active analogue thereof. Each member of this family includes seven cysteine residues clustered at the active C-terminal domain. Each member of the family can form dimers and bind to specific receptors, although the mode of dimerisation is different in each case. The proteins all adopt a unique three dimensional fold—the cysteine knot—that is characterised by an elongated β-strand and three disulphide bridges that display unusual connectivity. An example of a particularly suitable ligand which may be used in the composition of the invention and which belongs to the cysteine knot superfamily of proteins is spatzle protein derived from Drosophilia, or an active portion thereof, for example the C-terminal 106 amino acid peptide as described in Cell 76, 677-688. Other ligands from this family are described in TIBS 1998, July 23(7)(239-242). In a particularly preferred embodiment of the invention, the toll receptor ligand of the invention is a spatzle-like protein expressed during the larval stage of insects having such a larval life cycle, or a synthetic analogue thereof. Examples of such insects are Drosophila melanogaster and Lucilia sericata.

Compositions according to the invention which include toll receptor ligand precursors may further include a protease which is suitable for processing the toll receptor ligand precursor to form the active toll receptor ligand. Typically the protease will be a serine protease, for example a trypsin-like or chymotrypsin-like enzyme. A suitable trypsin-like protease is characterised in that:
(i) it is secreted by the organism Lucilia sericata;
(ii) it exhibits optimum proteolytic activity against FITC casein at a pH of 8.0 to 8.5;
(iii) it exhibits proteolytic ability against Tosyl-Gly-Pro-Arg-AMC but not against Suc-Ala-Ala-Phe-AMC;
(iv) its proteolytic activity against FITC-casein and Tosyl-Gly-Pro-Arg-AMC is inhibited by the serine protease inhibitors PMSF and APMSF; and
(v) it is bound by immobilised aminobenzamidine.
   A protease useful in the composition of the present invention exists, in nature, in the excretory/secretory (ES) secretions of the larvae of Lucilia sericata.

The larval ES secretions demonstrate a classical pH optimum of 8.0-8.5 when hydrolysing the fluorescent protein substrate fluorescein isothiocyanate-casein (FITC-casein). By pre-incubating the larval ES secretions, prior to monitoring the hydrolysis of FITC-casein, with the irreversible low molecular weight inhibitors 4-(amidinophenyl) methane sulphonyl fluoride (APMSF; an inhibitor for all trypsin-like serine proteases but not chymotrypsin-like serine proteinases) or with phenyl methanesulphonyl fluoride (PMSF; an inhibitor for all serine proteinases) it is shown that larval ES secretions have two types of serine proteinase activity; a trypsin-like activity and a chymotrypsin-like activity. The dual activity is confirmed by monitoring the hydrolysis of the fluorescent peptide substrates Tosyl-Gly-Pro-Arg-AMC (selective for trypsin-like proteinases) and Suc-Ala-Ala-Pro-Phe-AMC (selective for chymotrypsin-like proteinases), in which “AMC” represents 7-amino-4-methyl coumarin and “Suc” represents succinyl.

In addition to the predominant serine proteinase activity detected in the ES secretions of Lucilia sericata other less predominant activity is present. The presence of an aspartyl and metalloproteinase activity has been detected though no cysteinyl activity is shown. The aspartyl activity, shown by monitoring FITC-casein hydrolysis, is pronounced at pH 5.0 and is successfully inhibited by the class specific inhibitor pepstatin A. The metalloproteinase activity present is demonstrated by the ability of the ES secretions to hydrolyse a leucine aminopeptide, revealing the presence of an exopeptidase. Exopeptidases recognise free —NH₂ aminoacids in peptides. Leucine aminopeptide hydrolysis by Lucilia sericata ES is only inhibited by the Zn²⁺ chelator 1,10-phenanthroline, a classic metalloproteinsase inhibitor. This inhibition reflects the presence of an exopeptidase with a metalloproteinase enzymic nature.

The ES secretions have an α-amylase activity calculated to be about 0.88 units/litre. Additionally, phosphatase activity (hydrolysis of orthophosphoric monoester bond) is present in the larval ES secretions although this activity is approximately 50 times lower when compared to the proteinases. Lipase activity (hydrolysis of ester bonds found in fatty acid esters) is also identified. This lipase activity is not detected when the ES secretions are pre-incubated with the inhibitor PMSF, indicating that this hydrolysis is due to the serine proteinase in the secretions.

It can be concluded from our investigations that the predominant class of activity in the larval ES secretions is serine proteinase activity and that there are two types of serine proteinase activity present; one derived from a chymotryptic enzyme and one derived from a tryptic enzyme.

The processing protease may be obtained in substantially pure form from the crude ES secretions by a chromatographic procedure. The ES secretions are collected from the larvae of Lucilia sericata and are subjected to affinity chromatography using immobilised aminobenzamidine. Aminobenzamidine is a reversible inhibitor of trypsin-like serine proteinases. After collection of the “flow-through” material from the chromatographic procedure, i.e., the material which is not bound by the immobilised reagent, the enzyme which has been bound by the immobilised reagent may be eluted by the addition of free aminobenzamidine and collected separately.

The ligands of the invention, as described above, can be prepared synthetically and purified according to the usual routes of peptide synthesis and purification known in the art. The ligand may be protected against aminopeptidase activity to enhance activity and/or to prolong the period within which the ligand remains active in the wound area. Protection against aminopeptidase activity may, for example, be achieved by the amidation at COOH substitution in the ligand using a non-coded anomalous amino acid and/or CO—NH amide bond replacement by an isostere.

The ligands of the invention may be applied to a wound to induce a profile of growth factors conducive to healing. For instance, one or more ligands, either in a pure form or in a sterile carrier, can be sprinkled over the wound area or incorporated into a carrier to be applied to the wound. For instance, the ligand can be incorporated or encapsulated into a suitable material capable of delivering the ligand to a wound in a slow release or controlled release manner. An example of such a suitable material is poly(lactide-co-glycolide) or PLGA particles which may be formulated to release peptides in a controlled release manner. Alternatively, one or more ligands may be incorporated into a dressing to be applied over the wound. Examples of such dressings include staged or layered dressings incorporating slow-release hydrocolloid particles containing the wound healing material or sponges containing the wound healing material optionally overlayered by conventional dressings. Hydrocolloid dressings of the type currently in use, for example those available under the trademark “Granuflex”, may be modified to release the ligands to the wound.

The invention also relates to a method for treating a wound to promote healing thereof in a human or non-human which comprises applying to the wound a composition according to the invention. In a further aspect the invention provides a dressing for a wound which comprises a support carrying a composition according to the invention.

### DETAILED DESCRIPTION OF THE INVENTION

1. Isolation and Assay of the Processing Protease of the Invention

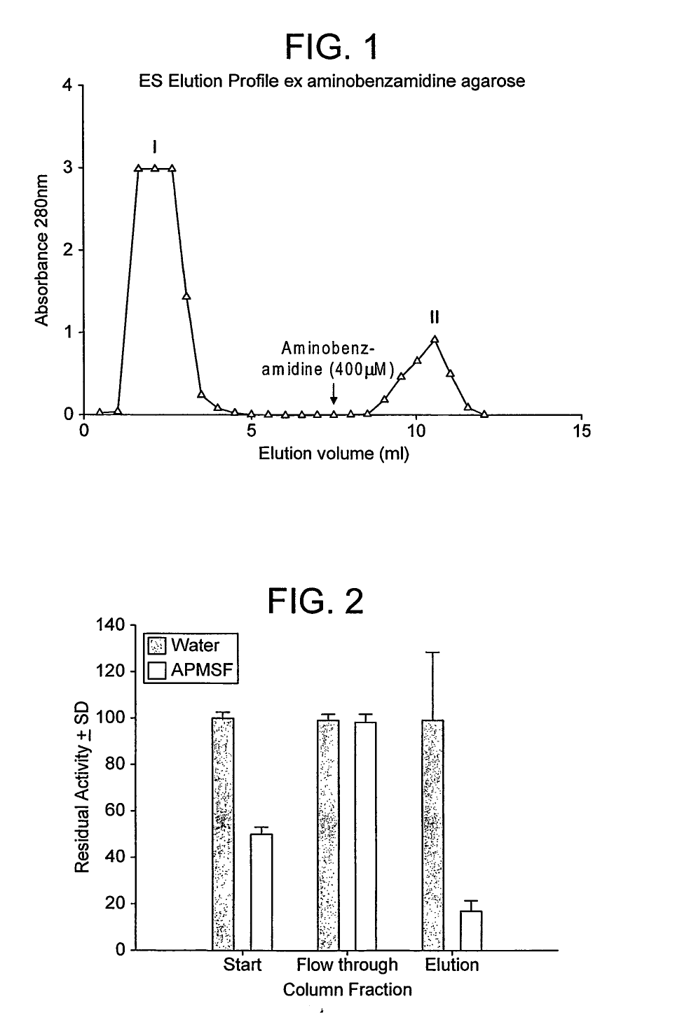

The trypsin-like serine proteinase was purified by affinity chromatography of Lucilia sericata ES on aminobenzamidine agarose. The column matrix (1 ml) was equilibrated with 20 ml of 0.025M Tris-HCl buffer pH 8.0 containing 0.5M NaCl. The crude ES (0.5 ml, 70 μg/ml protein) was diluted with an equal volume of buffer before application to the column. Fractions (0.5 ml) were collected throughout the chromatography. After washing with 6.5 times column volume of buffer to remove unbound protein, the free aminobenzamidine ligand (2 ml 400 μM) was used to elicit the elution of bound material. Absorbance readings of the fractions at 280 nm was used to establish the positions of the unbound (flow-through) and bound peaks which were then collected for assay. The elution profile is shown in FIG. 1

Aminobenzamidine agarose binds trypsin-like serine proteinases. Following application of larval enzyme secretions to the column, unbound material passed directly through and was collected as “flow-through” (peak 1). The addition of free aminobenzamidine to the column buffer elicited elution of the bound proteinase (peak 11). The unbound (flow-through) material contained proteinase activity unaffected by APMSF (possibly including a chrymotrypsin-like enzyme), whereas the activity in the aminobenzamidine elution peak was substantially abolished (80%) by APMSF, indicating purification of a trypsin-like serine proteinase activity. The residual activities of the column fractions are shown in FIG. 2.

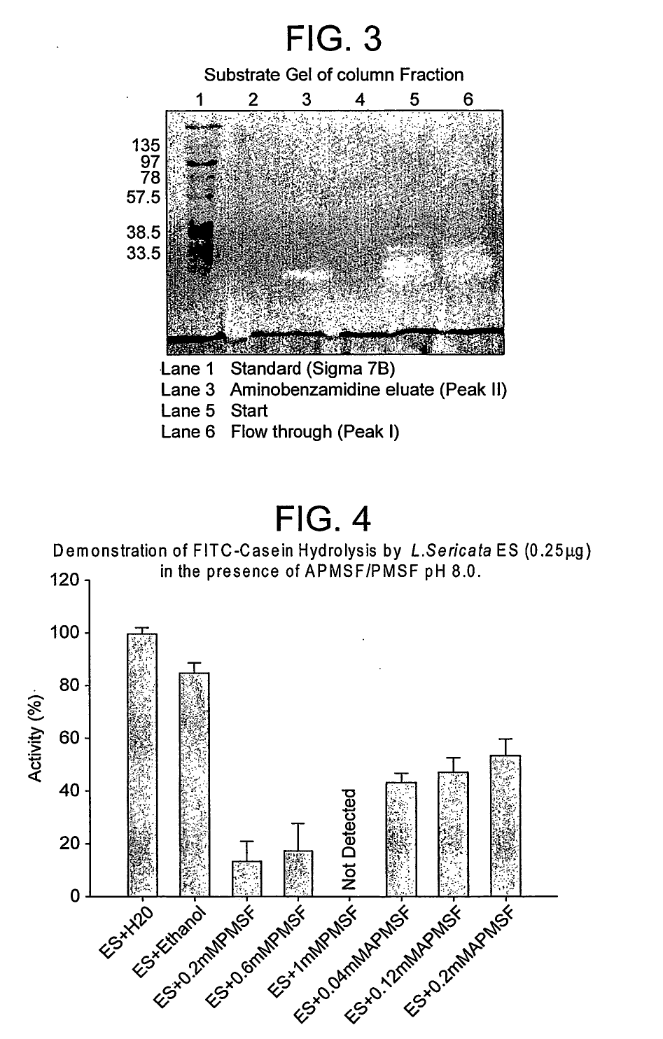

Column fractions were examined by electrophoresis in non-reducing SDS sample buffer (0.5M Tris-HCl pH 6.8 containing 4% SDS, 20% glycerol and 0.02% bromophenol blue) on 12% SDS polyacrylamide gels containing 0.1% human haemoglobin. SDS was removed by washing in 2.5% Triton X-100 (1 h) and distilled water (15 min). Proteolysis of the haemoglobin substrate in the gel by incubation at 37° C. in 0.1M Tris-HCl buffer pH 8.0 overnight produced clear bands revealed by protein staining in Coomassie Brilliant blue corresponding to the positions of proteinase enzymes (FIG. 3). The start and flow through fractions each showed several proteinase activities however the aminobenzamidine eluted a single band. Thus the trypsin-like enzyme previously identified in the aminobenzamidine-eluted fraction (FIG. 2) was shown to have molecular weight 25 Kda (FIG. 3).

2. Investigation of Proteolytic Behaviour of the Larval Enzyme (ES) With FITC-Casein

The activity of Lucilia sericata ES in FITC-casein hydrolysis at pH8 was investigated using different presentations of ES (0.25 kg) as follows:
A. ES+H₂O
B. ES+ethanol
C. ES pre-incubated with 0.2 mM PMSF
D. ES pre-incubated with 0.6 mM PMSF
E. ES pre-incubated with 1 mM PMSF
F. ES pre-incubated with 0.04 mM APMSF
G. ES pre-incubated with 0.12 mM APMSF
H. ES pre-incubated with 0.2 mM APMSF

The proteolytic activity of Lucilia sericata ES was inhibited following pre-incubation with the irreversible serine proteinase inhibitor PMSF. It was totally inhibited in the case where the ES had been pre-incubated with 1 mM PMSF. PMSF is dissolved in ethanol and the effect of the solvent on the activity of the ES was negligible. In contrast, approximately 50% of residual serine proteinase activity from ES was detected in the cases where the ES had been pre-incubated with the irreversible “trysin-like” specific inhibitor APMSF. Residual activity in the presence of APMSF indicates the presence of a chymotrypsin-like enzyme. The activity (%) values obtained were as follows:
A. 100%
B. 85.5%
C. 13.8%
D. 18%
E. 0%
F. 43.5%
G. 47%
H. 54%

These results are shown graphically in FIG. 4.

3. Investigation of the Proteolytic Activity of the Larval Enzyme (ES) Against Specific Substrates

The activity of Lucilia sericata ES (0.25 kg) against Tosyl-Gly-Pro-Arg-AMC (a) and against Suc-Ala-Ala-Phe-AMC (b) in the presence of APMSF and PMSF was investigated using different presentations of ES as follows:
(a)
   A. ES
   B. ES pre-incubated with 0.025 mM APMSF
   C. ES pre-incubated with 0.05 mM APMSF
   D. ES pre-incubated with 1 mM PMSF
(b)
   E. ES
   F. ES pre-incubated with 0.2 mM APMSF
   G. ES pre-incubated with 1 mM PMSF

The residual activity (%) values obtained were as follows:
(a)
   A. 100%
   B. 14.3%
   C. 3.6%
   D. 0%
(b)
   E. 100%
   F. 86.8%
   G. 1.3%

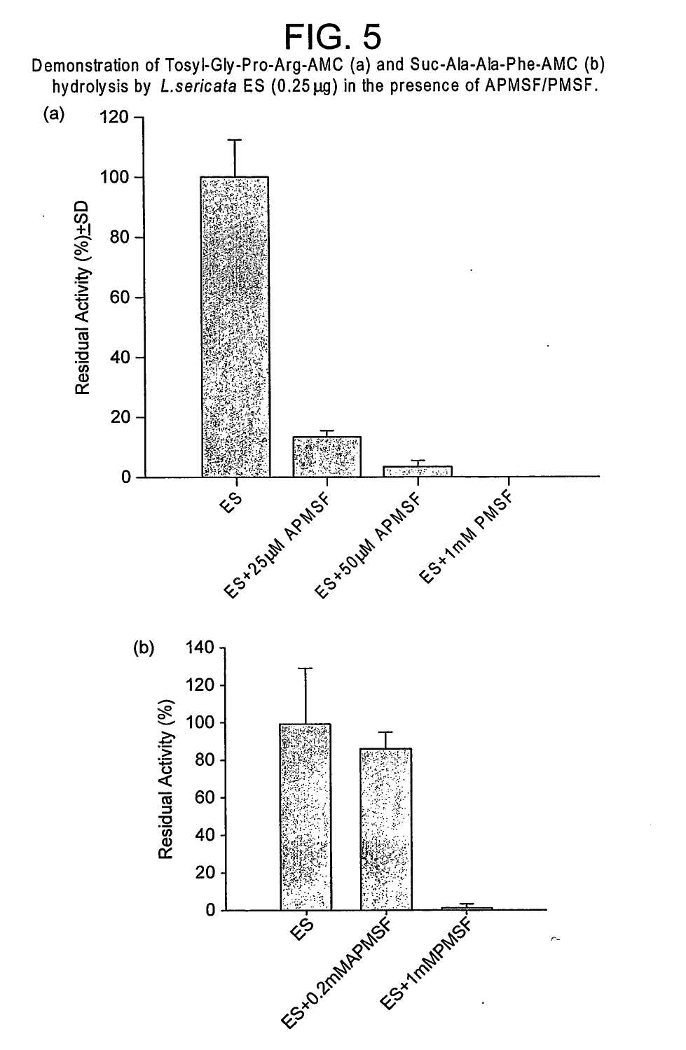

The results are shown graphically in FIG. 5.

The results for (a) reveal the “trypsin-like” serine proteinase activity present in Lucilia sericata ES. The hydrolysis of Tosyl-Gly-Pro-Arg-AMC (selective for the serine proteinases thrombin and plasmin) was inhibited by 1 mM PMSF and 0.05 mM APMSF. However, the hydrolysis of the chymotryptic substrate Suc-Ala-Ala-Phe-AMC by Lucilia sericata ES was only inhibited by PMSF (1 mM) and not by excess APMSF (which does not inhibit chymotrypsin). The results provide further evidence of the presence in ES of two different sub-classes of serine proteinase.

4. Ligands for Toll and Toll-Like Receptors

As mentioned above, ligands that may be used in the composition of the present invention may, according to a particular embodiment, be selected from constitutive and induced ligands for human toll receptors and may or may not be processed by proteases, such as serine proteases. A specific example is spatzle protein, a toll receptor ligand obtained from Drosophila melanogaster, in both its unprocessed and processed forms. Spatzle is described and characterized in Cell (1994), 76, 677-688.

Spatzle-like proteins (spatzle homologues or analogues) from different developmental stages of Lucilia sericata, may also be used as toll receptor ligands in the present invention. These may be identified using antibodies developed against the Drosophila spatzle protein. These may be purified from developmental stages of Lucilia sericata rich in spatzle-like proteins by extraction in physiological saline to give extracts that are then applied to antibody affinity chromatography columns to achieve purification. Spatzle-like proteins, thus identified, may be tested for their ability to engage toll receptors in human leucocytes. Human peripheral blood mononuclear (HPBM) cells may be co-cultured with the Spatzle-like protein from Lucilia sericata and the proliferation of the HPBM cells then measured using thymidine incorporation. In tandem, the ability of Lucilia Spatzle homologues or analogues to induce cytokine secretions (TNF-α) will be monitored alongside a known Toll ligand (LPS—bacterial lipopolysaccharide).

Ligands for toll-like receptors are described Cytokine and Growth Factor Reviews 11 (2000) 219-232.

Experimental

Studies were carried out to identify LPS-like activities in induced maggot haemolymph (using larvae of Lucilia sericata).

L. sericata larvae were grown on sterile liver/agar solution in the presence (induced) of or in the absence (non-induced) of Pseudomona aeruginosa.

Sterile larvae of L. sericata were obtained from Surgical Materials Testing Laboratory SMTL (Princess of Wales Hospital, Bridgend CF31 1RQ). The larvae were grown on medium described by Sherman (1995), comprising decomposed pig's liver and bacto-agar, sterilised by autoclaving in a closed container which allowed the exchange of gas and moisture between the interior and exterior of the container but which prevented the entry, into the container, of bacteria. A thin layer of nutrient medium, for the larvae, was provided in the base of the container.

Sterile first instar larvae (200) were suspended in 200 μl sterile phosphate buffered saline and transferred to the container. Growth was allowed under sterile conditions in a moisture chamber at 28° C. for ˜48 h to allow establishment of the larvae. Pseudomonas aeruginosa, mutant PAO P47, was inoculated into 10 ml Luria Bertani (LB) medium and grown overnight with shaking at 37° C. The container was inoculated with 1 ml (˜10⁸ viable counts) of the culture and the larvae allowed to grow in the presence of the bacteria.

The procedure described above was repeated but with the exception that no inoculation with P. aeruginosa culture was used.

After 48 hours incubation, the late 2^{nd} instar larvae from the procedures described above were processed separately as follows.

The larvae were removed from the liver-agar solutions and transferred into a sterile universal tube under sterile conditions. Maggots were washed with cold sterile PBS, then washed in 70% ethanol and finally dried on filter paper. The base of the larvae hooks was then sectioned using a sterile surgical blade. The haemolymph was then collected using a 201 pipette with sterile yellow Eppendorf tips and transferred into a pre-cooled Eppendorf tube containing 20 μg/ml of aprotinin (a protease inhibitor) and 40 μM phenylthiocarbamide (a melinisation inhibitor). After centrifugation at 15 000 g at 4° C. for 10 minutes, the supernatant was collected in a pre-cooled tube and kept in 80° C. until required. Gut contents did not contaminate haemolymph when this method was used, and haemolymph was not contaminated with P. aeruginosa, as adjudged by an overnight culture either in LB solution or plates.

The effect of induced and non-induced haemolymph supernatant (prepared as described above) on TNF-α release was tested using a ‘sandwich’ ELISA on human peripheral blood mononuclear cells (PBMCs) in comparison with LPS.

Blood specimens were obtained with consent from three healthy human volunteers (donor 1, 2 and 3). Human peripheral blood mononuclear cells (PBMC) from each of the three donors were isolated from heparinised whole blood by buoyant density centrifugation over Histopaque 1077 (Sigma, Poole, UK) at 600 g for 20 minutes. PBMC harvested from the intermediate layers were washed twice with RPMI 1640 medium and resuspended in AIM-V medium.

10⁵ PBMCs were then plated out onto a 96-well plate and incubated with 100 μl of increasing concentrations of non-induced/induced haemolymph and LPS from E. coli serotype 055:B5 as a positive control. After 24 hours incubation, cell supernatants were collected and added onto a 96-well plate pre-coated with a mouse anti-human TNF-α antibody. Serial dilutions of standard human TNF-α starting from 20 ng/ml were included in parallel. Prospective TNF-α was left overnight to capture and after three washes with 0.05% (v/v) PBS/Tween 20, the capture antibody was detected with the addition of a biotinylated mouse anti-human TNF-α antibody. After a final wash using 0.05% (v/v) PBS/Tween 20, streptavidin-horseradish peroxidase was added to the wells, developed for 10 minutes using tetramethyl-benzidine, as substrate, and the development read at 450 nm in a Dynex plate reader. All assays were carried out in duplicate.

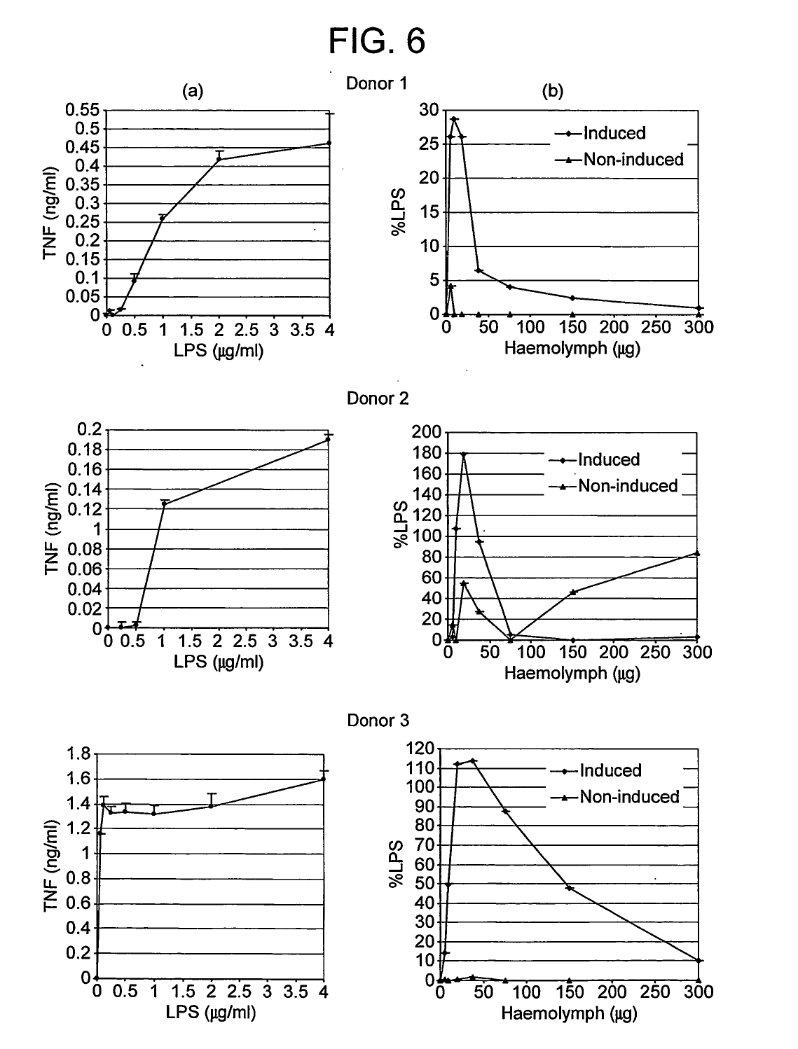

The results are shown graphically in FIG. 6. In FIG. 6, column (A) shows the plots obtained showing the relationship between TNF-α (ng/ml) detected against LPS (μg/ml) for the LPS treated PBMCs from each of the three donors. Column (B) shows the % TNF-α produced over the maximal LPS response (shown as % LPS) against haemolymph concentration for each of the haemolymph-treated PBMC samples. The plots in column (B) show the results for both the induced and the non-induced haemolymph. The study demonstrates that induced haemolymph stimulates TNF-α secretion from human PBMCs. It is important to mention that the haemolymph did not present any contamination with P. aeruginosa, as adjudged by an overnight culture either in LB solution or plates.

1. A composition for treatment of a wound to promote healing thereof in a human or non-human mammal which comprises an active amount of a toll receptor ligand, or a precursor thereof, and a suitable carrier.

2. The composition of claim 1 in which the toll receptor ligand or ligand precursor is a member of the cysteine knot superfamily of proteins, or an active analogue thereof.

3. The composition of claim 1 in which the toll receptor ligand or ligand precursor is an insect-derived protein, an active portion thereof, or an active analogue of either.

4. The composition of claim 3 in which the protein is derived from Drosophila melanogaster or Lucilia sericata.

5. The composition of claim 3 in which the active portion of the protein comprises a C-terminal 106 amino acid peptide.

6. The composition of claim 1 further comprising a protease which is suitable for processing a toll receptor ligand precursor to form an active toll receptor ligand.

7. The composition of claim 6 in which the protease is characterised in that:
(i) it is secreted by the organism Lucilia sericata;
(ii) it exhibits optimum proteolytic activity against FITC casein at a pH of 8.0 to 8.5;
(iii) it exhibits proteolytic ability against Tosyl-Gly-Pro-Arg-AMC but not against Suc-Ala-Ala-Phe-AMC;
(iv) its proteolytic activity against FITC casein and Tosyl-Gly-Pro-Arg-AMC is inhibited by the serine protease inhibitors PMSF and APMSF; and
(v) it is bound by immobilized and aminobenzamidine.

8. A method for treating a wound to promote healing thereof in a human or non-human which comprises applying to the wound a composition of claim 1.

9. A dressing for a wound which comprises a support carrying a composition of claim 1.
